(19) United States
(12) Patent Application Publication
(10) Pub. No.: US 2026/0000387 A1
(43) Pub. Date: Jan. 1, 2026

(54) ULTRASOUND DIAGNOSTIC APPARATUS

(71) Applicant: FUJIFILM Corporation, Tokyo (JP)

(72) Inventors: Jun YASUDA, Tokyo (JP); Tomohiko TANAKA, Tokyo (JP); Tetsuya YAMADA, Tokyo (JP)

(73) Assignee: FUJIFILM Corporation, Tokyo (JP)

(21) Appl. No.: 19/248,391

(22) Filed: Jun. 24, 2025

(30) Foreign Application Priority Data
Jun. 28, 2024 (JP) 2024-104920

(51) Int. Cl. A61B 8/00; A61B 8/06; A61B 8/14

(52) U.S. Cl. CPC A61B 8/5253; A61B 8/06; A61B 8/14; A61B 8/488; A61B 8/5223

(57) ABSTRACT

The information processing unit executes B-mode image generation processing of generating B-mode image data based on a reception signal generated by the transmission and reception unit, evaluation area setting processing of setting a blood flow evaluation area and a clutter evaluation area in a region where the B-mode image data is generated, wall filter processing on a Doppler reception signal generated by the transmission and reception unit, color Doppler processing of generating color mapping data for the B-mode image based on the Doppler reception signal subjected to the wall filter processing, evaluation processing of obtaining a pixel evaluation value for each of the blood flow evaluation area and the clutter evaluation area, and measurement condition setting processing of setting at least one of a transmission and reception condition in the transmission and reception unit or a characteristic of the wall filter processing according to each pixel evaluation value.

### CROSS REFERENCE TO RELATED APPLICATION

This application claims priority to Japanese Patent Application No. 2024-104920 filed on Jun. 28, 2024, which is incorporated herein by reference in their entireties including the specifications, claims, drawings, and abstracts.

### BACKGROUND OF THE INVENTION

### 1. Field of the Invention

The present disclosure relates to an ultrasound diagnostic apparatus, and particularly to transmission and reception conditions of ultrasonic waves and wall filter processing.

### 2. Description of the Related Art

There is a color Doppler mode as an operation mode of the ultrasound diagnostic apparatus. The color Doppler mode is an operation mode in which a blood flow velocity at an observation site is measured, and a color Doppler image in which a color corresponding to the blood flow velocity is overlaid to a B-mode image is displayed. In the measurement in the color Doppler mode, noise called clutter may appear on the color Doppler image depending on conditions such as a blood flow velocity at the observation site, a movement of a biological tissue, and characteristics of a wall filter.

Therefore, as shown in JP2009-005737A and JP1995-016227A (JP-H07-016227A), a technique of setting the characteristics of the wall filter according to the reception state of the ultrasonic wave has been suggested. JP2009-005737A describes that the characteristics of the wall filter are changed according to a pulse repetition frequency (PRF). JP1995-016227A (JP-H07-016227A) describes that the characteristics of the wall filter are changed according to the amplitude of the echo signal.

### SUMMARY OF THE INVENTION

The ultrasound diagnostic apparatus is used to observe various sites of a subject. In the related art, transmission and reception conditions of ultrasonic waves and wall filter characteristics are not always appropriate characteristics corresponding to the observation site, and noise such as clutter may easily occur depending on the observation site.

An object of the present disclosure is to suppress noise appearing in a color Doppler image.

An ultrasound diagnostic apparatus according to the present disclosure comprises a transmission and reception unit that transmits an ultrasonic wave to a subject through an ultrasound probe and receives a reflected wave reflected by the subject through the ultrasound probe; and an information processing unit, in which the information processing unit executes B-mode image generation processing of generating B-mode image data based on a reception signal generated by the transmission and reception unit, evaluation area setting processing of setting a blood flow evaluation area and a clutter evaluation area in a region where the B-mode image data is generated, wall filter processing on a Doppler reception signal generated by the transmission and reception unit, color Doppler processing of generating color mapping data for the B-mode image data based on the Doppler reception signal subjected to the wall filter processing, evaluation processing of obtaining a pixel evaluation value for each of the blood flow evaluation area and the clutter evaluation area, and measurement condition setting processing of setting at least one of a transmission and reception condition in the transmission and reception unit or a characteristic of the wall filter processing according to each pixel evaluation value.

In one embodiment, the information processing unit executes display processing of displaying a B-mode image based on the B-mode image data on a display device, and the evaluation area setting processing includes processing of setting the blood flow evaluation area and the clutter evaluation area according to an operation performed while the B-mode image is displayed.

In one embodiment, the measurement condition setting processing includes processing of setting at least one of the transmission and reception condition in the transmission and reception unit or the characteristic of the wall filter processing within a range determined according to an observation site.

In one embodiment, the information processing unit specifies the observation site according to a preset operation of setting each control parameter for each function of the ultrasound diagnostic apparatus.

In one embodiment, the information processing unit executes the evaluation processing a plurality of times while changing at least one of the transmission and reception condition or the characteristic of the wall filter processing, and searches for the transmission and reception condition and the characteristic of the wall filter processing in a case where a ratio of the pixel evaluation value for the blood flow evaluation area to the pixel evaluation value for the clutter evaluation area satisfies a predetermined condition, and the measurement condition setting processing includes processing of setting at least one of the transmission and reception condition or the characteristic of the wall filter processing such that the ratio satisfies the predetermined condition.

In addition, an ultrasound diagnostic apparatus according to the present disclosure comprises a transmission and reception unit that transmits an ultrasonic wave to a subject through an ultrasound probe and receives a reflected wave reflected by the subject through the ultrasound probe; and an information processing unit, in which the information processing unit executes wall filter processing on a Doppler reception signal generated by the transmission and reception unit, color Doppler processing of generating color mapping data for a B-mode image based on the Doppler reception signal subjected to the wall filter processing, and measurement condition setting processing of setting a transmission and reception condition in the transmission and reception unit and a characteristic of the wall filter processing according to an observation site.

In one embodiment, the information processing unit specifies the observation site according to a preset operation of setting each control parameter for each function of the ultrasound diagnostic apparatus.

In one embodiment, a cutoff frequency of the characteristic of the wall filter processing is set to a predetermined frequency with respect to the observation site.

According to the present disclosure, it is possible to suppress noise appearing in the color Doppler image.

### BRIEF DESCRIPTION OF THE DRAWINGS

FIG. 1 is a diagram showing a configuration of an ultrasound diagnostic apparatus according to a first embodiment.

FIG. 2 is a diagram showing Doppler shift frequency components and a high-pass filter characteristic.

FIG. 3 is a diagram showing an example of a B-mode image displayed on a display.

FIG. 4 is a diagram showing an ultrasound probe comprising a plurality of ultrasound transducers.

FIGS. 5A and 5B are diagrams showing an example of linear scanning with an ultrasound beam.

FIG. 6 is a diagram showing a configuration of an ultrasound diagnostic apparatus according to a second embodiment.

FIG. 7 is a diagram showing a configuration of an ultrasound diagnostic apparatus according to a third embodiment.

### DESCRIPTION OF THE PREFERRED EMBODIMENTS

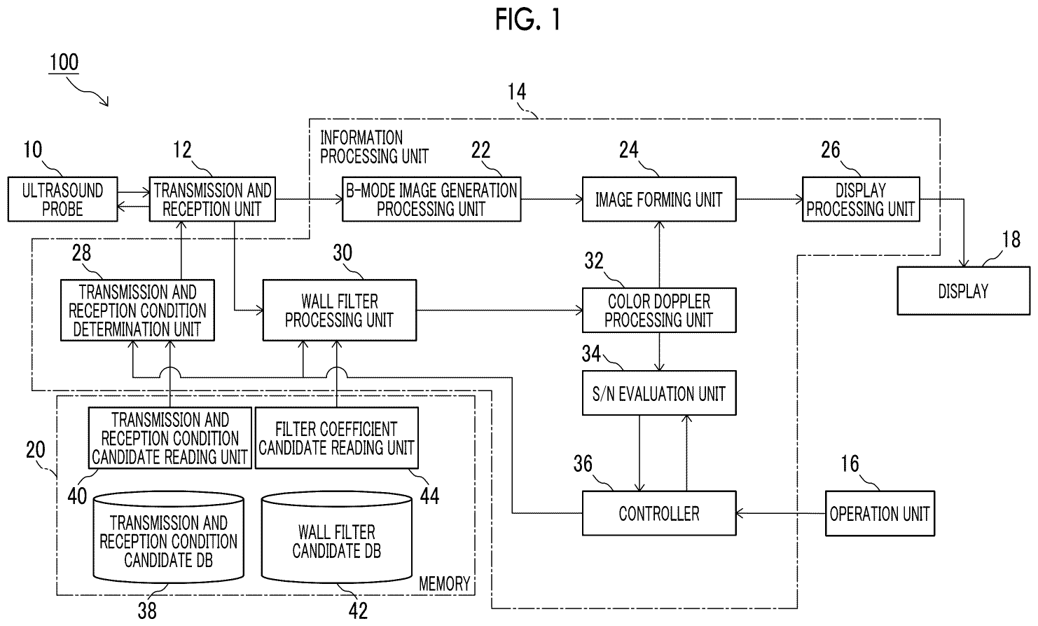

An embodiment of the present disclosure will be described with reference to the respective drawings. The same components shown in a plurality of drawings are designated by the same reference numerals to simplify the description thereof. FIG. 1 is a diagram showing a configuration of an ultrasound diagnostic apparatus 100 according to a first embodiment of the present disclosure. The ultrasound diagnostic apparatus 100 comprises an ultrasound probe 10, a transmission and reception unit 12, an information processing unit 14, an operation unit 16, a display 18, and a memory 20. The information processing unit 14 may comprise processors constituting a B-mode image generation processing unit 22, an image forming unit 24, a display processing unit 26, a transmission and reception condition determination unit 28, a wall filter processing unit 30, a color Doppler processing unit 32, an S/N evaluation unit 34, and a controller 36 by executing a program. The controller 36 performs overall control of an operation of the information processing unit 14. However, in FIG. 1, arrows indicating signals input to and output from the controller 36 are representative for simplification.

The operation unit 16 has a function as a human-machine interface and may comprise a mouse, a switch, a lever, a trackball, and the like. The operation unit 16 may constitute a touch panel together with the display 18. The controller 36 controls the ultrasound diagnostic apparatus 100 based on operation information output from the operation unit 16 by the operation of the user.

The ultrasound probe 10 comprises a plurality of ultrasound transducers. The transmission and reception unit 12 outputs a transmission signal to each ultrasound transducer provided in the ultrasound probe 10. Each ultrasound transducer generates an ultrasonic wave in response to the transmission signal. The transmission and reception unit 12 adjusts a delay time of the transmission signal output to each ultrasound transducer such that an ultrasound transmission beam is directed in a specific direction and a focus is formed. In addition, the transmission and reception unit 12 scans the subject with the ultrasound transmission beam by changing the delay time of the transmission signal output to each ultrasound transducer.

The ultrasonic wave transmitted to the subject is reflected by the biological tissue of the subject. The reflected waves generated in the biological tissue are received by each ultrasound transducer. Each ultrasound transducer converts the received ultrasonic wave into an electric signal and outputs the electric signal to the transmission and reception unit 12. The transmission and reception unit 12 adjusts a delay time of each electric signal such that electric signals output from each ultrasound transducer reinforce each other based on the reflected wave arriving from a direction of the ultrasound transmission beam, and adds and totals each of the electric signals. With such phase addition, a reception signal based on the reflected wave arriving from the direction of the ultrasound transmission beam is obtained, and an ultrasound reception beam is formed in the direction of the ultrasound transmission beam.

The transmission and reception unit 12 scans the ultrasound transmission beam and the ultrasound reception beam (hereinafter, the ultrasound transmission beam and the ultrasound reception beam are collectively referred to as an ultrasound beam) and outputs the reception signal obtained for each direction or each position of the ultrasound beam to the B-mode image generation processing unit 22 and the wall filter processing unit 30.

The B-mode image generation processing unit 22 generates B-mode image data based on the reception signals obtained for each direction or each position of the ultrasound beams, and outputs the B-mode image data to the image forming unit 24. The image forming unit 24 outputs the B-mode image data to the display processing unit 26, and the display processing unit 26 generates a video signal based on the B-mode image data as display processing and outputs the video signal to the display 18. A display device such as a liquid crystal panel or an organic EL panel, or a portable information processing device such as a tablet computer having a function as a display device may be used as the display 18. The display 18 displays a B-mode image based on the video signal.

An operation mode of the ultrasound diagnostic apparatus 100 includes a B-mode in which the B-mode image is displayed on the display 18 by the above-described processing, and a color Doppler mode. The color Doppler mode is an operation mode in which a blood flow velocity at the observation site is measured, and a color Doppler image in which a color corresponding to the blood flow velocity is overlaid to the B-mode image is displayed on the display 18.

The operation of the color Doppler mode is accompanied by the operation of the B-mode, and the operations of the B-mode and the color Doppler mode are executed by time-division. That is, a process in which the B-mode operation is executed in a certain time slot and the color Doppler mode operation is executed in the next time slot is repeated. In the color Doppler mode, the B-mode image data generated by the B-mode operation executed first is used.

The information processing unit 14 comprises a wall filter processing unit 30 and a color Doppler processing unit 32 as components for executing the operation in the color Doppler mode. In the following description, the reception signal output by the transmission and reception unit 12 to the wall filter processing unit 30 is referred to as a Doppler reception signal, which is distinguished from the reception signal output by the transmission and reception unit 12 to the B-mode image generation processing unit 22. The wall filter processing unit 30 performs high-pass filter processing as wall filter processing on the Doppler reception signal output from the transmission and reception unit 12, and outputs the Doppler reception signal to the color Doppler processing unit 32.

The color Doppler processing unit 32 obtains a Doppler shift frequency for each depth in an ultrasound beam direction in the subject based on a value of the Doppler reception signal corresponding to each depth in the ultrasound beam direction. Here, the Doppler shift frequency refers to a difference between the frequency of the transmission signal and the frequency of the Doppler reception signal. In the color Doppler mode, ultrasound pulses are repeatedly transmitted a plurality of times for each ultrasound beam direction, and reflected waves for each ultrasound pulse are received. The color Doppler processing unit 32 obtains a Doppler shift frequency for each depth in the ultrasound beam direction based on transmission and reception of the ultrasound pulses over a plurality of times. In a case where a direction of the blood flow is a direction toward the ultrasound probe 10, the Doppler shift frequency is positive, and in a case where the direction of the blood flow is a direction away from the ultrasound probe 10, the Doppler shift frequency is negative.

The color Doppler processing unit 32 obtains a Doppler shift frequency at each depth in the ultrasound beam direction for each direction or each position of the ultrasound beam scanned on the subject. As a result, the color Doppler processing unit 32 obtains a distribution of the Doppler shift frequencies in the scanning range of the ultrasound beam. The color Doppler processing unit 32 further generates color mapping data for coloring the B-mode image such that, for example, a first color is associated with a position where the Doppler shift frequency is positive and a second color is associated with a position where the Doppler shift frequency is negative, and outputs the color mapping data to the image forming unit 24.

The image forming unit 24 generates color Doppler image data and outputs the color Doppler image data to the display processing unit 26. Here, the color Doppler image data is data indicating a color Doppler image in which a color determined by the color mapping data is overlaid to each position on the B-mode image. The image forming unit 24 outputs the color Doppler image data to the display processing unit 26, and the display processing unit 26 generates a video signal based on the color Doppler image data as display processing and outputs the video signal to the display 18. The display 18 displays the color Doppler image based on the video signal.

In the color Doppler mode, a distribution of Doppler shift frequencies in the scanning range of the ultrasound beam is obtained based on the Doppler reception signal subjected to the high-pass filter processing. By performing the high-pass filter processing on the Doppler reception signal, the Doppler shift frequency component based on the movement of the organ such as the heart is reduced with respect to the Doppler shift frequency component based on the blood flow. As a result, the clutter appearing in the color Doppler image is suppressed. The clutter is noise based on the movement of the organ.

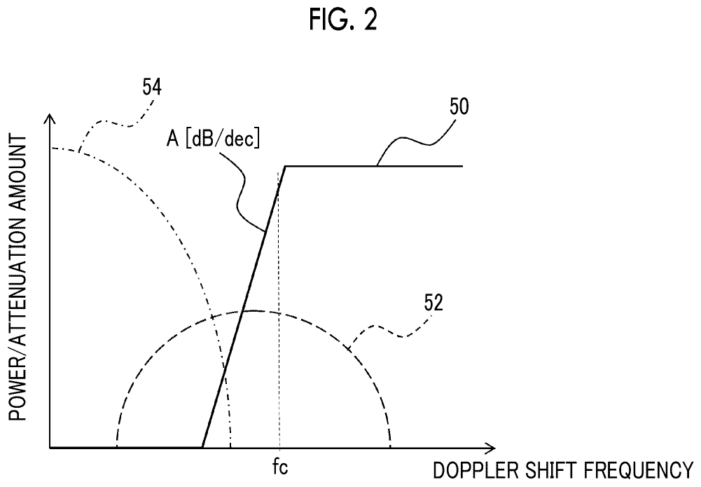

FIG. 2 shows the Doppler shift frequency components (52, 54) included in the Doppler reception signal and the high-pass filter characteristic 50 in the wall filter processing unit 30. The horizontal axis indicates the absolute value of the Doppler shift frequency, and the vertical axis indicates the power of the Doppler reception signal or the attenuation amount of the high-pass filter characteristic. In the high-pass filter characteristic 50 shown in FIG. 2, the attenuation amount is larger as the direction of the vertical axis is downward. In the following description, the high-pass filter characteristic in the wall filter processing unit 30 may be referred to as wall filter characteristics.

In the wall filter characteristics 50, in a frequency region lower than the cutoff frequency fc, the attenuation amount increases as the frequency approaches zero. In a frequency region higher than the cutoff frequency fc, the attenuation amount is smaller or zero as compared with that in the frequency range lower than the cutoff frequency fc. In the example shown in FIG. 2, the component 54 based on the movement of the organ among the Doppler shift frequency components extends from the zero frequency toward the high-frequency region, reaching up to the low-frequency region side of the component 52 based on the blood flow. The component 52 based on the blood flow overlaps the high-frequency region side of the component 54 based on the movement of the organ and reaches up to a higher frequency region side than the component 54. The cutoff frequency fc of the wall filter characteristics 50 is set to a position on a Doppler frequency axis at which the component 52 based on the blood flow is present on the higher frequency region side than the component 54 based on the movement of the organ. As a result, the component 52 based on the blood flow is dominant in the Doppler shift frequency component of the Doppler reception signal subjected to the wall filter processing.

The wall filter characteristics in the wall filter processing unit 30 in the present embodiment are defined by the cutoff frequency and the slope at the frequency equal to or lower than the cutoff frequency. The cutoff frequency is defined as a frequency at which the attenuation amount, increasing from the minimum value (for example, 0 dB) as it moves from the high-frequency region toward the low-frequency region, reaches 3 dB. The slope indicates, for example, an increase rate of the attenuation amount in a case where the frequency is decreased by 1/10, and is represented in units such as [dB/dec].

Here, as an example of the wall filter characteristics, a case where the vertical axis and the horizontal axis are decibel scales and the attenuation amount changes linearly is shown, but the wall filter characteristics may have various characteristics indicating high-pass filter characteristic.

The ultrasound diagnostic apparatus 100 may be used to observe various sites of a subject. In the ultrasound diagnostic apparatus of the related art, transmission and reception conditions of ultrasonic waves or wall filter characteristics are not always appropriate conditions or appropriate characteristics according to the observation site, and clutter may easily occur depending on the observation site. Therefore, in the present embodiment, the transmission and reception conditions of the ultrasonic waves and the wall filter characteristics are determined based on S/N evaluation processing described below.

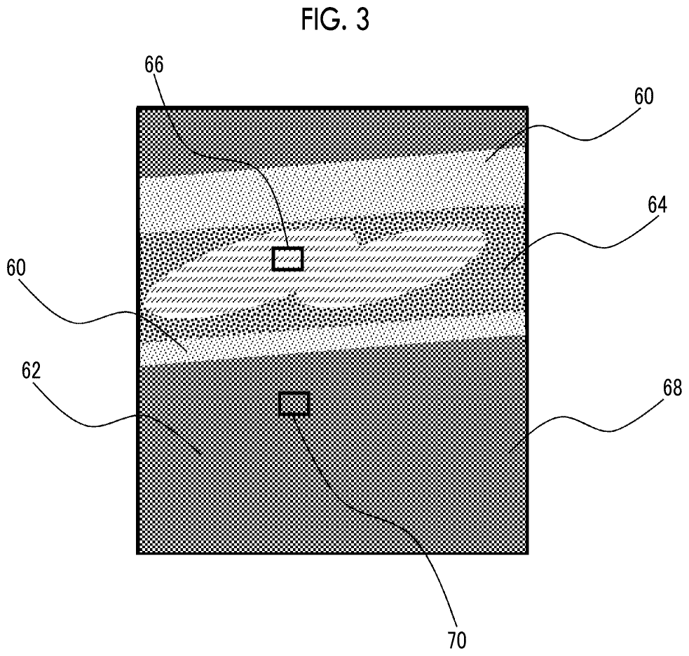

The S/N evaluation processing is executed in a case where the ultrasound diagnostic apparatus 100 is operating in the B-mode. FIG. 3 shows an example of the B-mode image displayed on the display 18. FIG. 3 shows a vascular wall 60 and a noise region 62. By the operation of the operation unit 16 by the user, the controller 36 sets, as evaluation area setting processing, a blood flow evaluation area 66 in the intravascular region 64 sandwiched between the two vascular walls 60, and sets a clutter evaluation area 70 in an extravascular region 68 outside the intravascular region 64. The blood flow evaluation area 66 and the clutter evaluation area 70 may be regions obtained by calculation in a virtual tomographic plane in the subject from which the B-mode image is acquired.

The B-mode image may be a set of a plurality of pixels, and the blood flow evaluation area 66 and the clutter evaluation area 70 may be a polygonal region, a substantially circular region, or a substantially elliptical region including a plurality of pixels. In the example shown in FIG. 3, each of the blood flow evaluation area 66 and the clutter evaluation area 70 is rectangular. The S/N evaluation unit 34 obtains a blood flow evaluation value as a pixel evaluation value of the blood flow evaluation area 66 and obtains a clutter evaluation value as a pixel evaluation value of the clutter evaluation area 70, based on the control of the controller 36. The pixel evaluation value is a value indicating a tendency of the size of the pixel in the area for which the pixel evaluation value is required. The blood flow evaluation value may be a statistical value such as an average value, a median value, or a mode value for a plurality of pixels included in the blood flow evaluation area 66. The clutter evaluation value may be a statistical value such as an average value, a median value, or a mode value for a plurality of pixels included in the clutter evaluation area 70.

The S/N evaluation unit 34 obtains an S/N evaluation value obtained by dividing the blood flow evaluation value by the clutter evaluation value as a ratio of the blood flow evaluation value to the clutter evaluation value, and outputs the S/N evaluation value to the controller 36. The controller 36 controls the transmission and reception condition determination unit 28 based on the S/N evaluation value to set the transmission and reception condition in a case where the ultrasound probe 10 transmits and receives the ultrasonic waves.

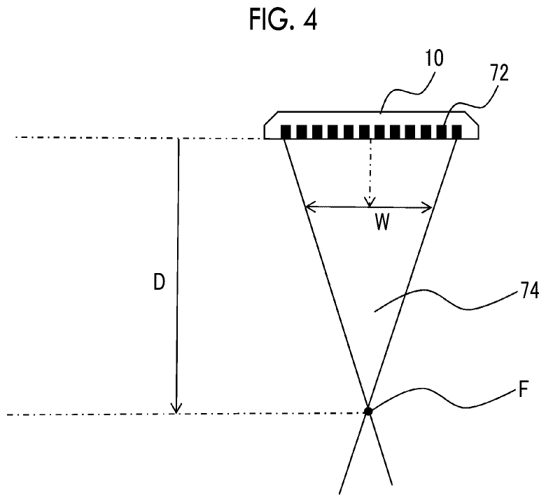

The transmission and reception conditions include an ultrasound distribution, a beam depth, a pulse repetition frequency, and the like. FIG. 4 schematically shows the ultrasound probe 10 comprising a plurality of ultrasound transducers 72. In the ultrasound probe 10, a plurality of ultrasound transducers 72 are arranged in the lateral direction (major axis direction). The beam depth D refers to a distance between the opening surface on which the ultrasound transducers 72 are arranged and the point where the focus F of the ultrasound beam 74 is formed. The ultrasound distribution may be defined as a maximum width W of a range in which ultrasonic waves having power exceeding a predetermined threshold value are distributed on a surface separated from the opening surface by a predetermined distance, for example. In addition, the pulse repetition frequency is defined as the reciprocal of a transmission interval of the ultrasound pulses in a case where the ultrasound pulses are repeatedly transmitted in the color Doppler mode.

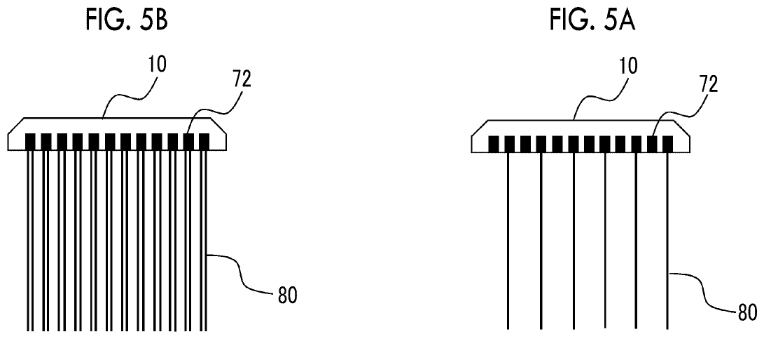

In addition, the transmission and reception condition includes an intensity of the ultrasonic wave transmitted from the ultrasound probe 10 and a scanning interval of the ultrasound transmission beam in a case of scanning with the ultrasound beam. The scanning interval may be an amount in units of an interval at which the ultrasound transducers 72 are arranged. FIGS. 5A and 5B show examples in which the ultrasound beam 80 extending in a direction perpendicular to the opening surface is linearly scanned in a direction in which the ultrasound transducers 72 are arranged. The scanning interval shown in FIG. 5A is twice the scanning interval shown in FIG. 5B.

A plurality of types of information for setting such transmission and reception conditions are stored in a transmission and reception condition candidate database (referred to as DB in the drawing) 38 stored in the memory 20. A transmission and reception condition candidate reading unit 40 provided in the memory 20 is hardware for reading out information from the transmission and reception condition candidate database 38. The transmission and reception condition determination unit 28 reads any of the plurality of types of information for setting the transmission and reception condition through the transmission and reception condition candidate reading unit 40 in the memory 20, and sets the transmission and reception condition in the transmission and reception unit 12.

In addition, the controller 36 may control the wall filter processing unit 30 in addition to the transmission and reception condition determination unit 28 based on the S/N evaluation value to set the wall filter characteristics. The wall filter characteristics are determined by, for example, the cutoff frequency and the slope as described above. By changing at least one of the cutoff frequency or the slope, the wall filter characteristics are changed. A plurality of types of filter tap coefficient groups for setting the wall filter characteristics are stored in a wall filter candidate database (referred to as DB in the drawing) 42 stored in the memory 20. A filter coefficient candidate reading unit 44 provided in the memory 20 is hardware for reading out information from the wall filter candidate database 42. The wall filter processing unit 30 comprises a digital filter of which characteristics are determined by inputting the filter tap coefficient group. The wall filter processing unit 30 reads any of the plurality of types of filter tap coefficient groups through the filter coefficient candidate reading unit 44 in the memory 20, and sets the wall filter characteristics of the wall filter processing unit 30.

The controller 36 may execute measurement condition setting processing of searching for the transmission and reception condition and the characteristics of the wall filter processing in a case where the S/N evaluation value satisfies a predetermined condition, and setting at least one of the transmission and reception condition or the characteristics of the wall filter processing such that the S/N evaluation value satisfies the predetermined condition. The predetermined condition may be a condition that the S/N evaluation value exceeds a predetermined threshold value or a condition that the S/N evaluation value is maximized.

The controller 36 repeatedly executes processing of acquiring the S/N evaluation value from the S/N evaluation unit 34 while changing at least one of the transmission and reception condition or the wall filter characteristics, for example, a plurality of times. The controller 36 causes the transmission and reception condition determination unit 28 and the wall filter processing unit 30 to set the transmission and reception condition and the wall filter characteristics in a case where the S/N evaluation value exceeds the predetermined threshold value, respectively. Then, the controller 36 causes the transmission and reception condition determination unit 28 and the wall filter processing unit 30 to maintain the transmission and reception condition and the wall filter characteristics in a case where the S/N evaluation value exceeds the predetermined threshold value.

As described above, the ultrasound diagnostic apparatus 100 comprises the transmission and reception unit 12 that transmits ultrasonic waves to the subject through the ultrasound probe 10 and receives reflected waves reflected by the subject through the ultrasound probe 10, and the information processing unit 14. The information processing unit 14 executes a program to configure the B-mode image generation processing unit 22, the image forming unit 24, the display processing unit 26, the transmission and reception condition determination unit 28, the wall filter processing unit 30, the color Doppler processing unit 32, the S/N evaluation unit 34, and the controller 36, and executes the following processing. That is, the information processing unit 14 executes the B-mode image generation processing, the wall filter processing, the color Doppler processing, the evaluation processing, and the measurement condition setting processing.

The B-mode image generation processing is processing of generating B-mode image data based on the reception signal generated by the transmission and reception unit 12. The evaluation area setting processing is processing of setting the blood flow evaluation area 66 and the clutter evaluation area 70 in the region in which the B-mode image data is generated. The wall filter processing is processing on the Doppler reception signal generated by the transmission and reception unit 12. The color Doppler processing is processing of generating color mapping data for the B-mode image based on the Doppler reception signal subjected to the wall filter processing. The evaluation processing is processing of obtaining a pixel evaluation value for each of the blood flow evaluation area 66 and the clutter evaluation area 70. The measurement condition setting processing is processing of setting at least one of the transmission and reception condition in the transmission and reception unit 12 or the characteristics of the wall filter processing according to each pixel evaluation value.

With the ultrasound diagnostic apparatus 100 according to the present embodiment, the S/N evaluation value is obtained for the blood flow evaluation area 66 and the clutter evaluation area 70 set by the user. Then, the transmission and reception condition and the wall filter characteristics are set based on the S/N evaluation value. As a result, the appropriate transmission and reception condition and the appropriate wall filter characteristics are set based on the blood flow evaluation area 66 and the clutter evaluation area 70 appropriately set based on the knowledge and the experience of the user, and the clutter appearing in the color Doppler image is suppressed.

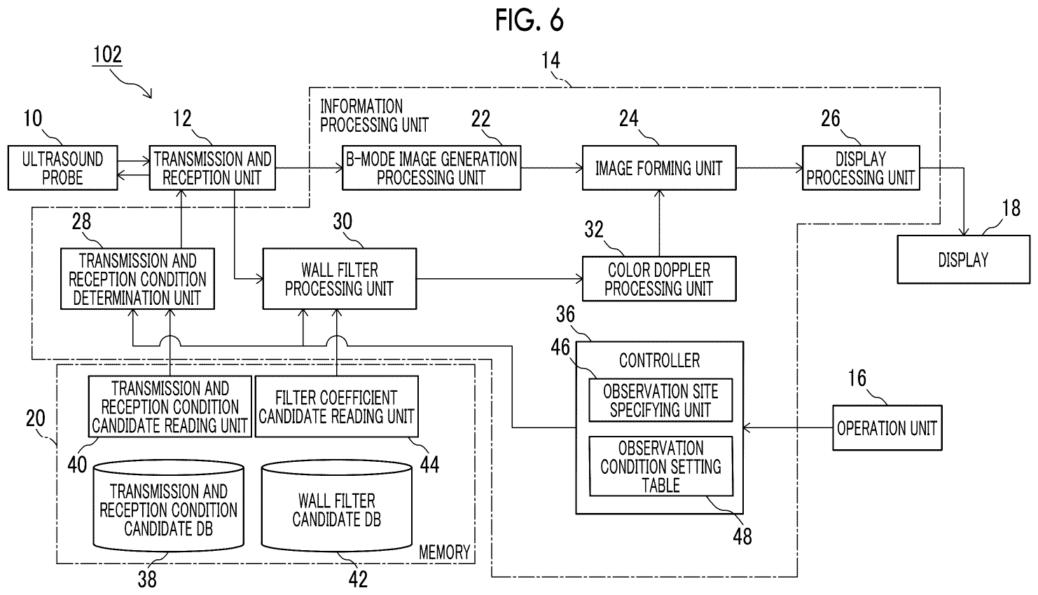

FIG. 6 shows a configuration of an ultrasound diagnostic apparatus 102 according to a second embodiment. The ultrasound diagnostic apparatus 102 is different from the ultrasound diagnostic apparatus 100 in that the controller 36 comprises an observation site specifying unit 46 and an observation condition setting table 48.

The controller 36 executes a preset that sets each control parameter for each function of the ultrasound diagnostic apparatus 102. By activating the preset for a certain function, for example, each control parameter is set to a default value (standard value) for the function. For a certain control parameter, the display processing unit 26 displays an input format image for inputting the control parameter on the display 18. The control parameter is set by the operation of the user on the operation unit 16 for the input format image. The preset includes various diagnosis categories such as obstetric early stage, thyroid diagnosis, abdominal diagnosis, and chest diagnosis, and the diagnosis category is selected by the operation of the user on the operation unit 16.

Examples of the control parameters include a depth (diagnosis range) of an observation range, a gain with respect to a reception signal, a time gain control, a focus, and an echo enhancement level. The time gain control is a control parameter for adjusting a state in which the gain is increased with the elapse of the reception time. The focus is a control parameter indicating a degree to which the ultrasound beam is converged. The echo enhancement level is a control parameter indicating a degree of emphasizing a contour of the B-mode image.

The observation site specifying unit 46 in the controller 36 may specify the observation site in the subject according to the preset operation. In this case, the observation site specifying unit 46 specifies a diagnostic site such as a heart, a carotid artery, a lower limb blood vessel, and a liver, based on, for example, the diagnosis category for which the preset has been executed and each control parameter set in the preset.

In general, there are appropriate transmission and reception conditions and wall filter characteristics for forming a color Doppler image for each diagnostic site of the subject. That is, since an observation position and the observation range differ depending on the diagnostic site, an appropriate depth of focus and an appropriate ultrasound distribution differ depending on the observation site. In addition, the tendency of the magnitude of the blood flow velocity differs depending on the diagnostic site, and the tendency of the spectrum of the Doppler shift frequency component for the blood flow differs depending on the diagnostic site. Therefore, appropriate wall filter characteristics differ depending on the diagnostic site.

Therefore, the controller 36 executes measurement condition setting processing of setting the transmission and reception condition and the characteristics of the wall filter processing in the transmission and reception unit 12 according to the observation site. The controller 36 stores in advance the observation condition setting table 48 in which information indicating predetermined appropriate transmission and reception condition and the wall filter characteristics is associated with information for specifying a diagnostic site. The controller 36 refers to the observation condition setting table 48 to acquire the setting information. The setting information indicates appropriate transmission and reception condition and the wall filter characteristics for the diagnostic site specified by the observation site specifying unit 46.

The controller 36 controls the transmission and reception condition determination unit 28 to set the transmission and reception condition in the transmission and reception unit 12 to the transmission and reception condition indicated by the setting information. The transmission and reception condition determination unit 28 reads information that is suitable for the setting information from the transmission and reception condition candidate database 38, from among a plurality of types of information, through the transmission and reception condition candidate reading unit 40 in the memory 20, and sets the transmission and reception condition in the transmission and reception unit 12 to the transmission and reception condition indicated by the setting information.

The controller 36 controls the wall filter processing unit 30 to set the wall filter characteristics to the characteristics indicated by the setting information. The wall filter processing unit 30 reads information that is suitable for the setting information from the wall filter candidate database 42, from among the plurality of types of filter tap coefficient groups, through the filter coefficient candidate reading unit 44 in the memory 20, and sets the wall filter characteristics in the wall filter processing unit 30.

According to such processing, the appropriate transmission and reception condition and the wall filter characteristics are rapidly set for the diagnostic site.

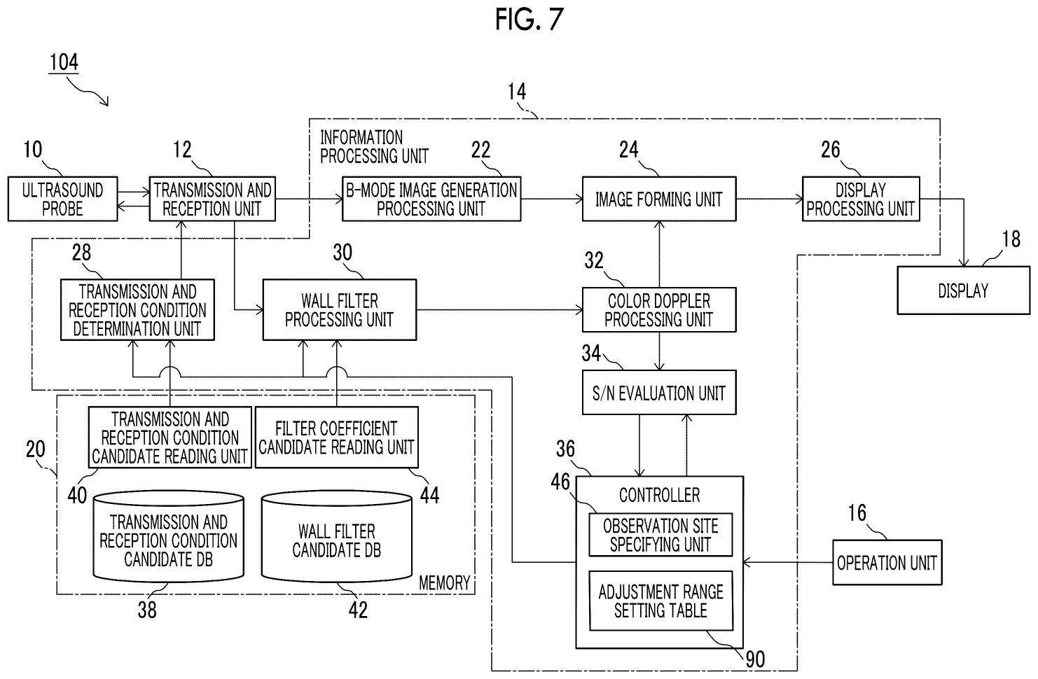

FIG. 7 shows an ultrasound diagnostic apparatus 104 according to a third embodiment. The ultrasound diagnostic apparatus 104 is obtained by adding an observation site specifying unit 46 and an adjustment range setting table 90 to the controller 36 of the ultrasound diagnostic apparatus 100 according to the first embodiment.

As described above, the observation site specifying unit 46 specifies the diagnostic site and generates information for specifying the diagnostic site. The adjustment range setting table 90 is a table in which information indicating the adjustment range of each of the transmission and reception condition and the wall filter characteristics is associated with the information specifying the diagnostic site. The adjustment range of the transmission and reception condition may be represented by a lower limit value and an upper limit value of each numerical value representing the transmission and reception condition. The adjustment range of the transmission and reception condition includes an appropriate value of each numerical value representing the transmission and reception condition. The adjustment range of the transmission and reception condition may be represented by, for example, lower limit values and upper limit values of each of the ultrasound distribution, the beam depth, the pulse repetition frequency, the scanning interval of the ultrasound transmission beam, and the intensity of the ultrasonic wave transmitted from the ultrasound probe 10.

The adjustment range of the wall filter characteristics is represented by the lower limit value and the upper limit value of each numerical value representing the wall filter characteristics. The adjustment range of the wall filter characteristics includes an appropriate value of each numerical value representing the wall filter characteristics. The adjustment range of the wall filter characteristics may be represented by, for example, lower limit values and upper limit values of each of the cutoff frequency, the slope, and the like.

The controller 36 refers to the adjustment range setting table 90 to acquire the adjustment range of the transmission and reception condition and the adjustment range of the wall filter characteristics for the diagnostic site specified by the observation site specifying unit 46.

The controller 36 controls the transmission and reception condition determination unit 28 based on the S/N evaluation value within the adjustment range of the transmission and reception condition, and sets the transmission and reception condition in a case where the ultrasound probe 10 transmits and receives the ultrasonic wave. In addition, the controller 36 controls the wall filter processing unit 30 based on the S/N evaluation value within the adjustment range of the wall filter characteristics, and sets the wall filter characteristics.

The controller 36 acquires the S/N evaluation value from the S/N evaluation unit 34 while changing at least one of the transmission and reception condition or the wall filter characteristics, for example, within the adjustment range of the transmission and reception condition and within the adjustment range of the wall filter characteristics. The controller 36 causes the transmission and reception condition determination unit 28 and the wall filter processing unit 30 to set the transmission and reception condition and the wall filter characteristics in a case where the S/N evaluation value exceeds the predetermined threshold value, respectively. Then, the controller 36 causes the transmission and reception condition determination unit 28 and the wall filter processing unit 30 to maintain the transmission and reception condition and the wall filter characteristics in a case where the S/N evaluation value exceeds the predetermined threshold value.

According to such processing, the processing of setting the transmission and reception condition and the wall filter characteristics is performed within the adjustment range of the transmission and reception condition and within the adjustment range of the wall filter characteristics. Since the adjustment range in a case of setting appropriate transmission and reception condition and the adjustment range in a case of setting appropriate wall filter characteristics are limited, processing of setting the transmission and reception condition and the wall filter characteristics is performed rapidly.

What is claimed is:

1. An ultrasound diagnostic apparatus comprising:
a transmission and reception unit that transmits an ultrasonic wave to a subject through an ultrasound probe and receives a reflected wave reflected by the subject through the ultrasound probe; and
an information processing unit,
wherein the information processing unit executes
B-mode image generation processing of generating B-mode image data based on a reception signal generated by the transmission and reception unit,
evaluation area setting processing of setting a blood flow evaluation area and a clutter evaluation area in a region where the B-mode image data is generated,
wall filter processing on a Doppler reception signal generated by the transmission and reception unit,
color Doppler processing of generating color mapping data for the B-mode image data based on the Doppler reception signal subjected to the wall filter processing,
evaluation processing of obtaining a pixel evaluation value for each of the blood flow evaluation area and the clutter evaluation area, and
measurement condition setting processing of setting at least one of a transmission and reception condition in the transmission and reception unit or a characteristic of the wall filter processing according to each pixel evaluation value.

2. The ultrasound diagnostic apparatus according to claim 1,
wherein the information processing unit executes display processing of displaying a B-mode image based on the B-mode image data on a display device, and
the evaluation area setting processing includes processing of setting the blood flow evaluation area and the clutter evaluation area according to an operation performed while the B-mode image is displayed.

3. The ultrasound diagnostic apparatus according to claim 1,
wherein the measurement condition setting processing includes processing of setting at least one of the transmission and reception condition in the transmission and reception unit or the characteristic of the wall filter processing within a range determined according to an observation site.

4. The ultrasound diagnostic apparatus according to claim 3,
wherein the information processing unit specifies the observation site according to a preset operation of setting each control parameter for each function of the ultrasound diagnostic apparatus.

5. The ultrasound diagnostic apparatus according to claim 2,
wherein the measurement condition setting processing includes processing of setting at least one of the transmission and reception condition in the transmission and reception unit or the characteristic of the wall filter processing within a range determined according to an observation site.

6. The ultrasound diagnostic apparatus according to claim 5,
wherein the information processing unit specifies the observation site according to a preset operation of setting each control parameter for each function of the ultrasound diagnostic apparatus.

7. The ultrasound diagnostic apparatus according to claim 1,
wherein the information processing unit
executes the evaluation processing a plurality of times while changing at least one of the transmission and reception condition or the characteristic of the wall filter processing, and
searches for the transmission and reception condition and the characteristic of the wall filter processing in a case where a ratio of the pixel evaluation value for the blood flow evaluation area to the pixel evaluation value for the clutter evaluation area satisfies a predetermined condition, and
the measurement condition setting processing includes processing of setting at least one of the transmission and reception condition or the characteristic of the wall filter processing such that the ratio satisfies the predetermined condition.

8. The ultrasound diagnostic apparatus according to claim 2,
wherein the information processing unit
executes the evaluation processing a plurality of times while changing at least one of the transmission and reception condition or the characteristic of the wall filter processing, and
searches for the transmission and reception condition and the characteristic of the wall filter processing in a case where a ratio of the pixel evaluation value for the blood flow evaluation area to the pixel evaluation value for the clutter evaluation area satisfies a predetermined condition, and
the measurement condition setting processing includes processing of setting at least one of the transmission and reception condition or the characteristic of the wall filter processing such that the ratio satisfies the predetermined condition.

9. An ultrasound diagnostic apparatus comprising:
a transmission and reception unit that transmits an ultrasonic wave to a subject through an ultrasound probe and receives a reflected wave reflected by the subject through the ultrasound probe; and
an information processing unit,
wherein the information processing unit executes
wall filter processing on a Doppler reception signal generated by the transmission and reception unit,
color Doppler processing of generating color mapping data for a B-mode image based on the Doppler reception signal subjected to the wall filter processing, and
measurement condition setting processing of setting a transmission and reception condition in the transmission and reception unit and a characteristic of the wall filter processing according to an observation site.

10. The ultrasound diagnostic apparatus according to claim 9,
wherein the information processing unit specifies the observation site according to a preset operation of setting each control parameter for each function of the ultrasound diagnostic apparatus.

11. The ultrasound diagnostic apparatus according to claim 9,
wherein a cutoff frequency of the characteristic of the wall filter processing is set to a predetermined frequency with respect to the observation site.

12. The ultrasound diagnostic apparatus according to claim 10,
wherein a cutoff frequency of the characteristic of the wall filter processing is set to a predetermined frequency with respect to the observation site.
